(19) United States
(12) Patent Application Publication
(10) Pub. No.: US 2011/0002895 A1
(43) Pub. Date: Jan. 6, 2011

(54) COMPOSITION FOR AUTOTRANSPLANTATION OR ALLOTRANSPLANTATION USING DENTAL PULP STEM CELL, AND USE OF THE COMPOSITION

(76) Inventors: Minoru Ueda, Nagoya-shi (JP); Yoichi Yamada, Nagoya-shi (JP); Sayaka Nakamura, Nagoya-shi (JP); Kenji Ito, Nagoya-shi (JP); Yudai Nishino, Nagoya-shi (JP)

Correspondence Address: EDWARDS ANGELL PALMER & DODGE LLP, P.O. BOX 55874, BOSTON, MA, 02205, US

(21) Appl. No.: 12/734,911

(22) PCT Filed: Dec. 3, 2008
(86) PCT No.: PCT/JP2008/071975

§ 371 Date: Sep. 2, 2010

(30) Foreign Application Priority Data
Dec. 5, 2007 (JP) 2007-315195
Mar. 6, 2008 (JP) 2008-057017

(51) Int. Cl. A61K 35/32; C12N 5/00

(52) U.S. Cl. 424/93.7; 435/325

(57) ABSTRACT

The object is to provide a novel use application of a dental pulp stem cell collected from a deciduous tooth or a permanent tooth. Disclosed is a composition for autotransplantation or allotransplantation, which is characterized by comprising a dental pulp stem cell collected from a deciduous tooth or a permanent tooth.

### TECHNICAL FIELD

The present invention relates to a composition which is utilized in reparation or regeneration (reconstruction) of a tissue. More specifically, the present invention relates to a composition which is utilized for autotransplantation or allotransplantation using a dental pulp stem cell, and use thereof (such as an operative method using the composition).

### BACKGROUND ART

The most important factor for cell treatment or regenerative medicine is a cell. For example, bone marrow cells and cord blood stem cells have been used for the treatment of leukemia. In order to seek smooth utilization of these cells, bone marrow banks and cord blood banks have been established, but the amount of cells ensured is not necessarily sufficient.

Meanwhile, it was recently found that strong stem cells are present in permanent teeth and milk teeth (deciduous teeth), which has gathered an attention (for example, see Patent Document 1 and Non-patent Document 1). Specifically, cells in deciduous teeth have following advantages as compared to stem cells in bone marrow or cord blood: (1) the cells have high growth ability (can grow cells by culture), (2) the cells have high differentiation ability (comprise cells which form bone, cartilage, nerve, blood vessel and the like), and (3) the cells are readily collected (deciduous teeth naturally drop off when a child reaches 6 to 10 years of age). The deciduous tooth stem cells having such excellent features are considered to play an important role in cell treatment and regenerative medicine in the future, and studies are moving forward all over the world. On the other hand, presence of similar stem cells was confirmed in a dental pulp of a permanent tooth (for example, see Patent Document 1). Furthermore, the inventors have reported that a dentine was formed by mixing dental pulp cells with a carrier matrix (see Patent Document 2).

### [Patent Document 1] WO2006/010600

### [Patent Document 2] JP-A No. 2004-201612

### [Patent Document 3] JP-A No. 2006-230316

### [Patent Document 4] JP-A No. 2006-265221

[Non-patent Document 1] Miura M. et al., SHED: Stem cells from human exfoliated deciduous teeth, PNAS, May 13, 2003, vol. 100, no. 10, 5807-5812

### DISCLOSURE OF THE INVENTION

### Problems to be Solved by the Invention

Under the above-mentioned background, the present invention aims at providing novel use of dental pulp stem cells collected from deciduous teeth or permanent teeth.

### Means to Solve the Problems

The present invention has been completed as a result of intensive studies aiming at solving the above-mentioned problem, and is as follows.

[1] A composition for autotransplantation or allotransplantation, comprising a dental pulp stem cell from a deciduous tooth or a permanent tooth.

[2] The composition for autotransplantation or allotransplantation according to [1], wherein the dental pulp stem cell is CD13 positive, CD29 positive, CD44 positive, CD73 positive, CD105 positive, CD146 positive, CD14 negative, CD34 negative and CD45 negative cell.

[3] The composition for autotransplantation or allotransplantation according to [1] or [2], which is characterized by comprising the dental pulp stem cell by about 1.0×10³ to about 1.0×10⁸ cells/ml.

[4] The composition for autotransplantation or allotransplantation according to any one of [1] to [3], comprising a platelet-rich plasma.

[5] The composition for autotransplantation or allotransplantation according to any one of [1] to [3], which is characterized by that the dental pulp stem cell is suspended in a physiological saline or a phosphate-buffered physiological saline.

[6] The composition for autotransplantation or allotransplantation according to any one of [1] to [5], comprising a cytokine.

[7] The composition for autotransplantation or allotransplantation according to any one of [1] to [6], wherein the composition is used for regeneration of a bone tissue, a cartilage tissue, a nerve tissue, a skin tissue, a hair tissue, a periodontal tissue or a blood vessel tissue.

[8] A method for regenerating a tissue, wherein the composition for autotransplantation or allotransplantation according to any one of [1] to [7] is injected in, embedded in, filled in or applied on a tissue-defective site.

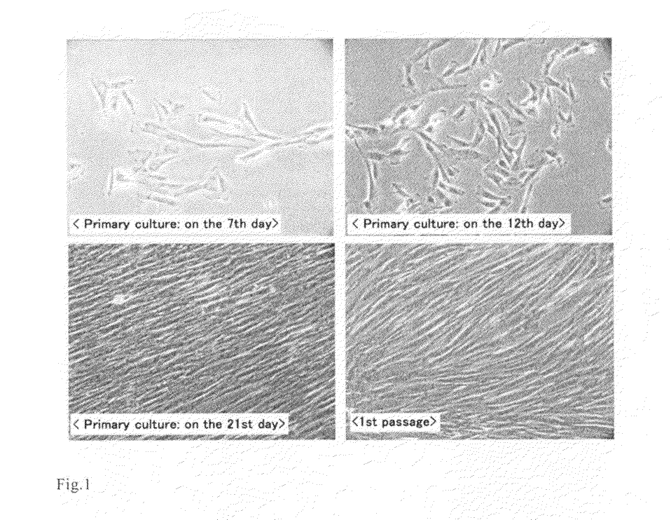

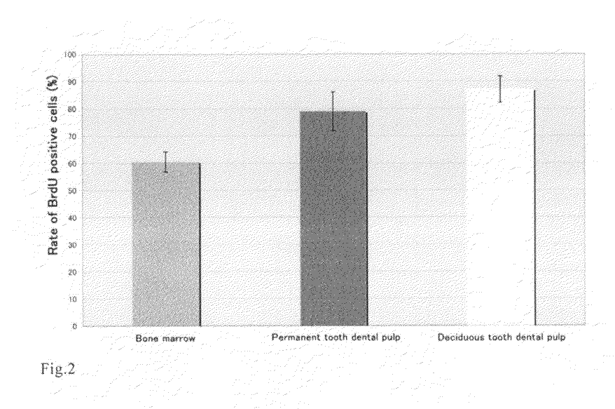

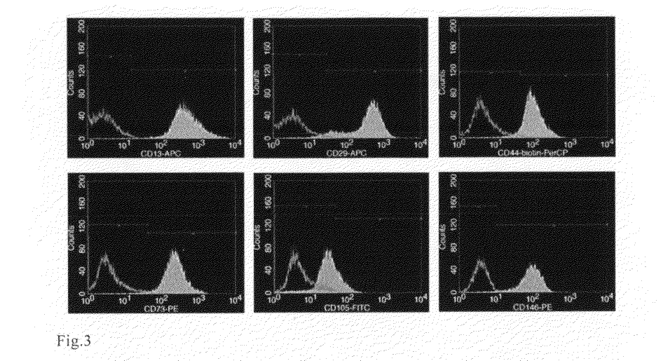

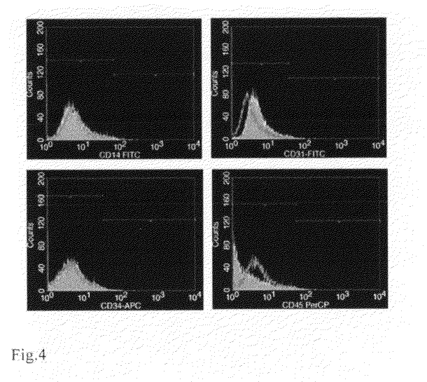

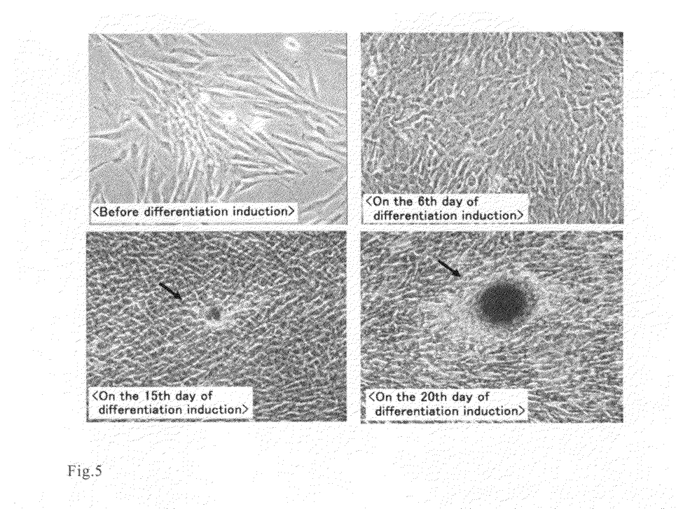

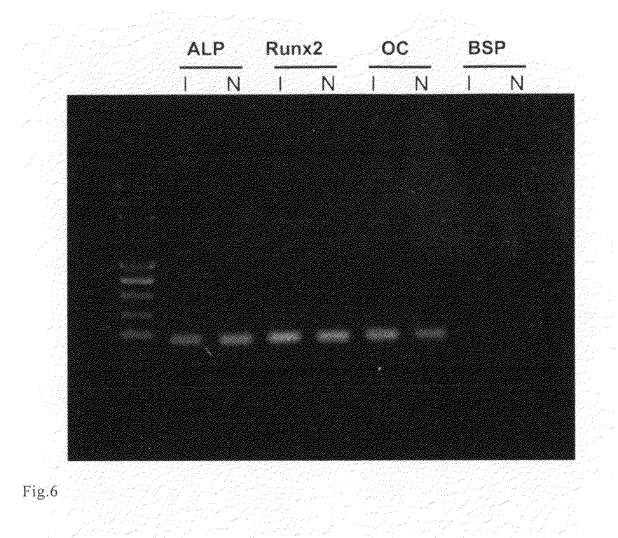

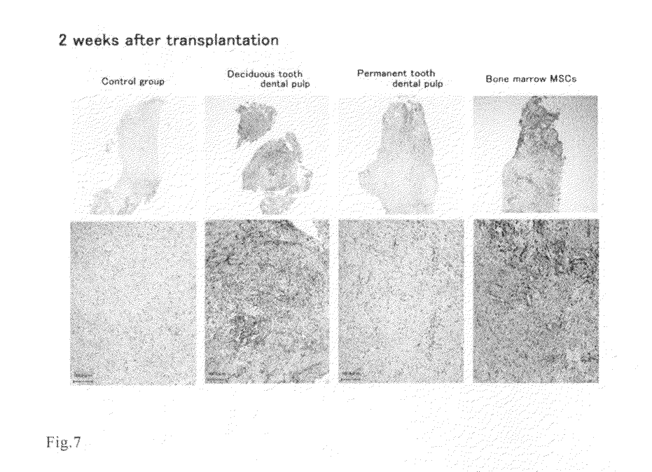

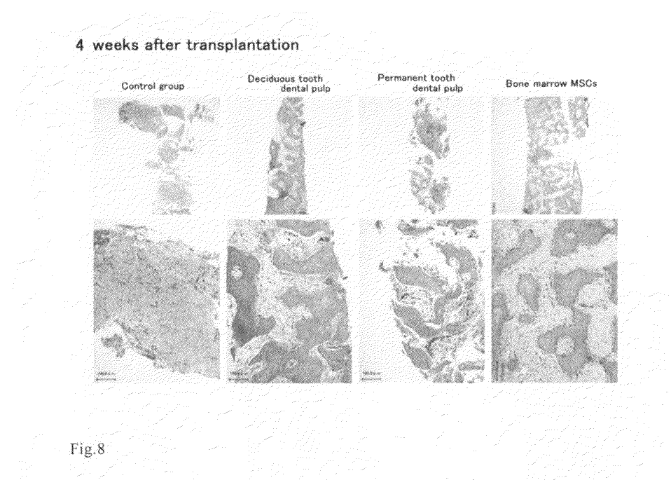

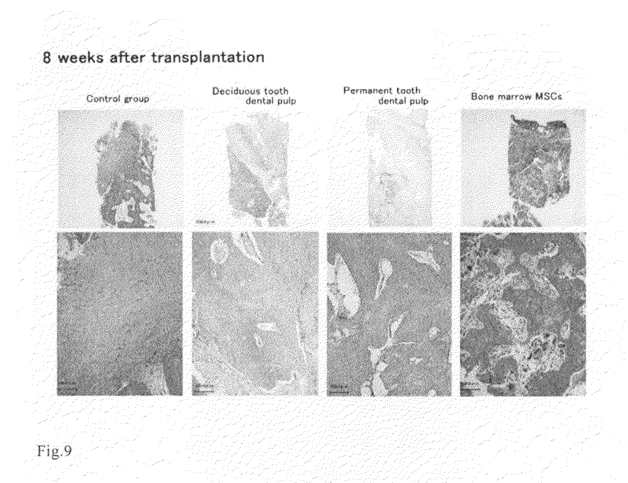

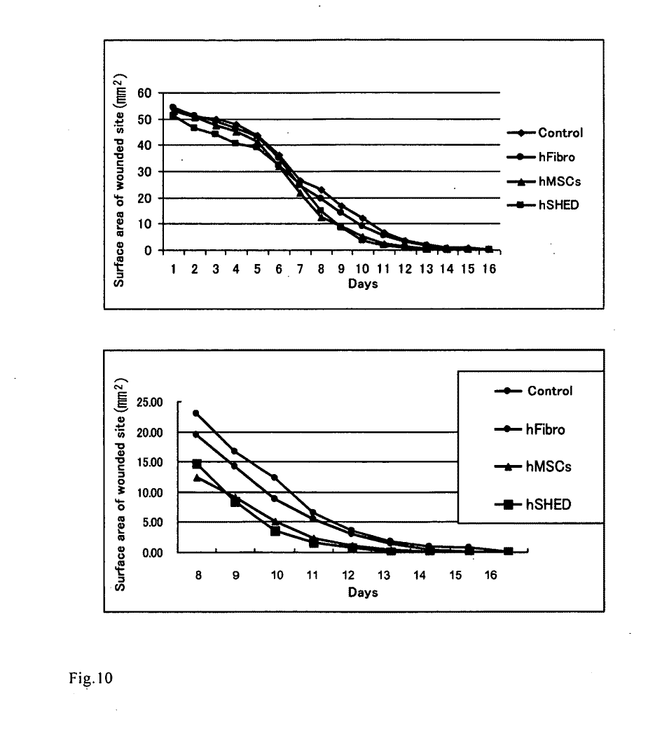

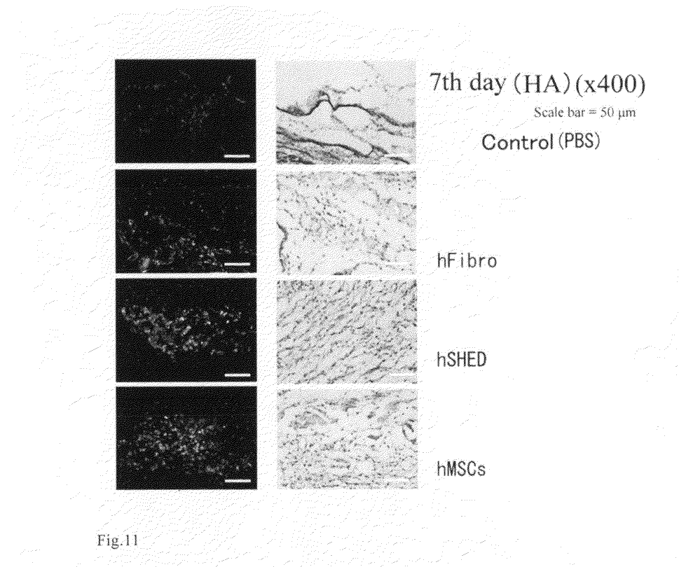

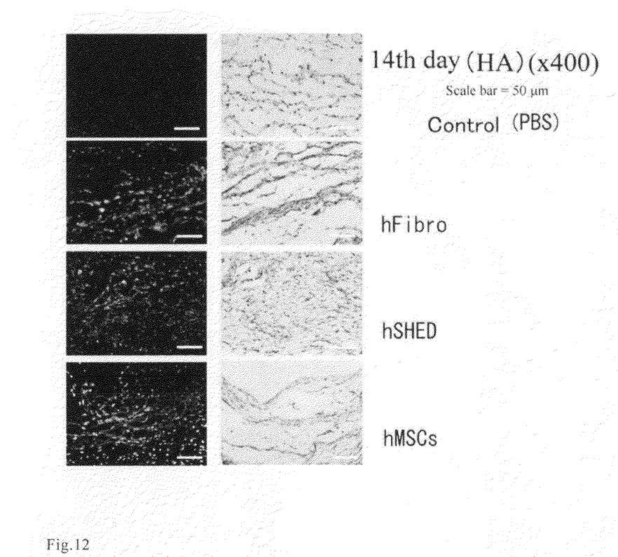

### BRIEF DESCRIPTION OF THE DRAWINGS

[FIG. 1] A microscopic image showing the growth ability of deciduous tooth dental pulp stem cells. Upper left: the 7^{th} day of primary culture, upper right: the 12^{th} day of primary culture, lower left: the 21^{st} day of primary culture, lower right: the 1^{st} passage.

[FIG. 2] Comparison of the growth rates of bone marrow-derived stem cells, deciduous tooth dental pulp stem cells and permanent tooth dental pulp stem cells. It is apparent that the deciduous tooth dental pulp stem cells (right) had higher growth ability than permanent tooth dental pulp stem cells (center). As a control, the growth rate of human mesenchymal stem cells (hMSC) is shown (left).

[FIG. 3] The result of the flow cytometry (FCM) using deciduous tooth dental pulp stem cells as samples. It is apparent that the cells are CD13 positive, CD29 positive, CD44 positive, and CD73 positive, CD105 positive and CD146 positive.

[FIG. 4] The result of flow cytometry (FCM) using deciduous tooth dental pulp stem cells as samples. It is apparent that the cells are CD14 negative, CD31 negative, CD34 negative and CD45 negative.

[FIG. 5] A microscopic image showing differentiation of the deciduous tooth dental pulp stem cells to osteoblasts. Upper left: before differentiation induction, upper right: the 6^{th} day of differentiation induction, lower left: the 15^{th} day of differentiation induction, lower right: the 20^{th} day of differentiation induction.

[FIG. 6] Expression of an osteoblast marker gene in the deciduous tooth dental pulp stem cells after differentiation induction. Expression of an osteoblast marker is observed.

[FIG. 7] Starting from the left, the stained image of the transplanted site on the 2^{nd} week (control), the tissue image of the transplanted site on the 2^{nd} week after transplantation of the mixture of PRP/deciduous tooth dental pulp stem cells, the tissue image of the transplanted site on the 2^{nd} week after transplantation of the mixture of PRP/permanent tooth dental pulp stem cells, and the tissue image of the transplanted site on the 2^{nd} week after transplantation of the mixture of PRP/bone marrow stem cells.

[FIG. 8] Starting from the left, the stained image of the transplanted site on the 4^{th} week (control), the tissue image of the transplanted site on the 4^{th} week after transplantation of the mixture of PRP/deciduous tooth dental pulp stem cells, the tissue image of the transplanted site on the 4^{th} week after transplantation of the mixture of PRP/permanent tooth dental pulp stem cells, and the tissue image of the transplanted site on the 4^{th} week after transplantation of the mixture of PRP/bone marrow stem cells.

[FIG. 9] Starting from the left, the stained image of the transplanted site on the 8^{th} week (control), the tissue image of the transplanted site on the 8^{th} week after transplantation of the mixture of PRP/deciduous tooth dental pulp stem cells, the tissue image of the transplanted site on the 8^{th} week after transplantation of the mixture of PRP/permanent tooth dental pulp stem cells, and the tissue image of the transplanted site on the 8^{th} week after transplantation of the mixture of PRP/bone marrow stem cell.

[FIG. 10] Change of the surface area of the wounded site over days after transplantation. Human deciduous tooth dental pulp stem cells (hSHED), human bone marrow-derived mesenchymal stem cells (hMHCs), or human oral mucosa-derived fibroblasts (hFibro) were each transplanted on a wounded site formed on the dorsal region of a nude mouse, and the changes in the surface area of the wounded site were compared. The case where PBS was transplanted was considered as a control. The upper column is a graph showing the change from the 1^{st} day to 16^{th} day after transplantation. The lower column is a graph extracting from the 8^{th} day to 16^{th} day after transplantation.

[FIG. 11] Immunofluorescence staining targeting hyarulonic acid. The fluorescence surface area stained image (×400) on the 7^{th} day after transplantation is shown on the left line. The right line is the hematoxylin-eosin stained image of the same (×400). Starting from the top, a control group (PBS), a human oral mucosa-derived fibroblast (hFibro)-transplanted group, a human deciduous tooth dental pulp stem cell (hSHED)-transplanted group, and a human bone marrow-derived mesenchymal stem cell (hMHCs)-transplanted group. The scale bar is 50 μm.

[FIG. 12] Fluorescence immunostaining targeting hyarulonic acid. The fluorescence surface area stained image (×400) on the 14^{th} day after transplantation is shown in the left line. The right line is the hematoxylin-eosin stained image of the same (×400). Starting from the top, control group (PBS), human oral mucosa-derived fibroblast (hFibro)-transplanted group, human deciduous tooth dental pulp stem cell (hSHED)-transplanted group, and human bone marrow-derived mesenchymal stem cell (hMHCs)-transplanted group. The scale bar is 50 μm.

### BEST MODE OF CARRYING OUT THE INVENTION

### (Composition for Autotransplantation or Allotransplantation)

The first aspect of the present invention relates to a composition for autotransplantation or allotransplantation. The composition for autotransplantation or allotransplantation of the present invention is characterized by comprising dental pulp stem cells from deciduous teeth or permanent teeth. Preferably, dental pulp stem cells from deciduous teeth is used. This is because the growth ability of the cells is higher than that of dental pulp stem cells from permanent teeth and also because it is considered that the cells have higher differentiation ability. On the other hand, easiness of collection is also an advantage of use of the deciduous tooth dental pulp stem cells.

The dental pulp stem cells used for the present invention are CD13 positive, CD29 positive, CD44 positive, CD73 positive, CD105 positive, CD146 positive, CD14 negative, CD34 negative and CD45 negative cells.

One embodiment of the present invention comprises platelet-rich plasma besides cell components. Herein, “platelet-rich plasma”, PRP refers to plasma containing abundance of platelets. In other words, it refers to plasma containing concentrated platelet. PRP can be prepared by subjecting blood collected to centrifugation in accordance with, for example, the method by Whitman et al. (Dean H. Whitman et al.: J Oral Maxillofac Surg, 55, 1294-1299 (1997)). PRP is known to include an abundance of growth factors such as Platelet-derived Growth Factor (PDGF), Transforming growth factor β1 (TGF-β1), Transforming growth factor β2 (TGF-β2), and the like (Jarry J. Peterson: Oral surg Oral Med Oral Pathol Oral Radiol Endod, 85, 638-646 (1998)).

One embodiment of the present invention comprises a cytokine besides cell components. Preferably, a cytokine is included besides platelet-rich plasma. As the cytokine, for example, BMP, PDGF, bFGF and the like are used. Two or more kinds of cytokines may be used in combination.

When PRP and/or a cytokine are used, regeneration effect can be enhanced. Furthermore, PRP is also effective for the preparation of flowability or viscosity. Namely, the composition for autotransplantation or allotransplantation of the present invention can be prepared in the form of a gel which is suitable for transplantation, by using PRP (see the following “Column of Preparation Method”).

Under the condition that the effect expected for the composition for autotransplantation or allotransplantation of the present invention (namely, regeneration of the tissue on the applied site) is retained, additional use of other components is not impeded. The components which may be additionally used in the present invention are listed below.

### (1) Inorganic Bioabsorbable Material and Organic Bioabsorbable Material

The kinds of the inorganic bioabsorbable material are not particularly limited, but it is possible to use a material selected from the group consisting of β-tricalcium phosphate (β-TCP), α-tricalcium phosphate (α-TCP), tetracalcium phosphate, octacalcium phosphate, and amorphous calcium phosphate. These materials can be used singly. In addition, the combination of arbitrarily selected two or more materials may be used. Preferably, it is possible to use either β-TCP or α-TCP and the combination of them at arbitrary ratio may be used. More preferably, β-TCP is used as an inorganic bioabsorbable material. The inorganic bioabsorbable material can be obtained by a well-known method. Furthermore, commercially available inorganic bioabsorbable material can be also used. As the β-TCP, for example, one manufactured by OLYMPUS CORPORATION can be used.

(25-1) It is preferable that the inorganic bioabsorbable material has a powdery form having a particle diameter such that the composition of the present invention has a fluidity when it is used.
(25-2) The powdery inorganic bioabsorbable material can be prepared by breaking and crushing an inorganic bioabsorbable material that has been processed so that it has an appropriate size. It is preferable that the average particle diameter of the inorganic bioabsorbable material is 0.5 μm to 50 μm. It is further preferable that the inorganic bioabsorbable material having the average particle diameter of 0.5 μm to 10 μm is used. It is yet further preferable that the inorganic bioabsorbable material having the average particle diameter of 1 μm to 5 μm is used. It is also possible to use the combination of a plurality of inorganic bioabsorbable materials having different particle diameters.
(25-3) It is preferable that the content of the inorganic bioabsorbable material is 30 wt. % to 75 wt. % with respect to the entire composition.
(25-4) Note here that the fluidity of the composition of the present invention can be adjusted by the particle diameter and content of the inorganic bioabsorbable material and by appropriately adjusting the both, a desired fluidity can be obtained. Furthermore, when a thickener mentioned below is added, the fluidity can be also adjusted by the addition amount of the thickener.

As the organic bioabsorbable material, hyaluronic acid, collagen, fibrinogen (for example, Bolheal (registered trademark)), and the like, can be used.

### (2) Gelation Material

(27-1) For example, the composition of the present invention can be constructed by adding thrombin and calcium chloride. When such materials are added, thrombin acts on the fibrinogen in the PRP, so that fibrin is generated. Then, due to the coagulation action of fibrin, viscosity is increased. The kinds of the gelation materials are not particularly limited and a material that increases the viscosity by acting the component in the PRP as described above, or a material having a thickening effect by itself can be appropriately selected and used.
(27-2) Furthermore, in addition to the above-mentioned gelation materials, a second gelation material, which acts after application (after transplantation) so as to change the fluidity (viscosity) of the composition of the present invention, can be used. With such a configuration, the composition is easily transplanted because it has an appropriate fluidity when it is used, and the composition has an improved fixation at the application site because it has an increased viscosity after application. Thus, the tissue can be repaired or regenerated efficiently. Furthermore, it is not necessary to shape the material in a shape of the site to be applied in advance, thus increasing the versatility.
(27-3) As the gelation material, a material having a high biocompatibility is preferably used. In addition to the above-mentioned examples, hyaluronic acid, collagen or fibrin glue, or the like, can be used. Various kinds of collagen can be selected and used as hyaluronic acid or collagen. However, it is preferable to employ collagen suitable for application object of the composition (tissue to which the composition is applied) in accordance with the present invention. When the object is to regenerate the bone tissue, for example, type I collagen can be used. It is preferable that the collagen to be used has a solubility (acid soluble collagen, alkali soluble collagen, enzyme soluble collagen, and the like).

### (3) Thickener

It is also possible to adjust the fluidity of the composition of the present invention by adding a thickener. As the thickener, thickening polysaccharides such as sodium alginate, glycerine, vaseline, or the like, can be used. From the viewpoint of safety and/or bone forming ability, it is preferable to use a thickener having a high biocompatibility and having a bioabsorbable property or a biodegradability. By adding glycerine or the like, an antifreezing effect can be obtained.

### (4) Solvent

The composition of the present invention may include an aqueous solvent. An example of the aqueous solvent can include sterile water, a physiological saline solution, a buffer solution such as a phosphate solution, and the like. In addition, the prepared cells may also be suspended in a physiological saline or PBS (phosphate-buffered physiological saline) to form the composition of the present invention (which does not comprise other components such as PRP) and applied on an affected area.

### (5) Others

The composition of the present invention may include a stabilizer, a preservative, a pH regulator, and the like, in addition to the above-mentioned components. Furthermore, the composition may also include a growth factor, in particular, a bone inducing factor (BMP).

### (Method for Application)

The composition for autotransplantation or allotransplantation of the present invention is used for regeneration of a bone tissue, a cartilage tissue, a nerve tissue, a skin tissue, a hair tissue, a periodontal tissue or a blood vessel tissue by autotransplantation or allotransplantation. As the method for application, injecting in, embedding in, filling in or applying on a tissue-defective site can be adopted. When the composition is prepared into a gel form having suitable flowability, it can be applied by a convenient means such as filling, injection or application. Furthermore, a gel form is highly versatile since it can readily be filled in the application site by using a syringe or the like (the gel form can also be applied without opening the wounded site), and it is not necessary to be formed into the shape of the tissue-deleted site in advance.

Meanwhile, there are a technique comprising using mesenchymal cells separated from bone marrow, peripheral blood or cord blood for wound healing (JP-A No. 2006-230316 (Patent Document 3)) and a technique for applying fibroblast cell growth factor (FGF-1) to skin care (JP-A No. 2006-265221 (Patent Document 4)). These techniques have high invasiveness, and there are many unclear points with respect to effective cell sources, established culture methods and applications to treatment. Furthermore, the specifics of the treatment effect are also unclear. On the other hand, it has been clarified in recent years that there are stem cells (deciduous tooth dental pulp stem cells and permanent tooth dental pulp stem cells) in a dental pulp of a deciduous tooth and a permanent tooth, but the effect on wound healing has not been sufficiently studied, and the specifics thereof are unclear. Under such circumstances, the present inventors have studied the treatment effect of deciduous tooth dental pulp stem cells by using wound healing models (Examples mentioned below). As a result, it was found that deciduous tooth dental pulp stem cells exhibit a treatment effect equal to that of mesenchymal stem cells which are considered to promote wound healing. Based on this finding, in a preferable embodiment of the present invention, the composition of the present invention is utilized for wound healing (acceleration of wound healing, prevention of keloid, prevention of scar and/or improvement of skin type).

### (Method for Preparation of Dental Pulp Stem Cells)

Hereinafter an example of a procedure for preparing dental pulp stem cells is shown. In this preparation method, (1) collection of a dental pulp, (2) enzyme treatment and seeding of the cells, (3) selective culturing of adherent cells, (4) differentiation induction, and (5) collection of cells are performed in this order, whereby dental pulp stem cells which are compatible to a tissue to be regenerated is prepared. For example, where a bone tissue is to be regenerated, dental pulp stem cells which have been differentiation-induced to bone cells are prepared. Meanwhile, in the present specification, dental pulp stem cells for which differentiation to a specific cell lineage has been induced are also referred to as “dental pulp stem cells”. Therefore, in an embodiment of the composition for autotransplantation or allotransplantation of the present invention, the cells for which differentiation to a specific cell lineage has been induced are included as the “dental pulp stem cells”.

Hereinafter the steps in the preparation method are explained.

### (1) Collection of Dental Pulp

A deciduous tooth which naturally dropped off (or an extracted deciduous tooth, or a permanent tooth) is soaked in a solution of chlorohexidine or isodine, and recovered in a culture medium. Then, the deciduous tooth (or extracted deciduous tooth, or permanent tooth) is divided where necessary using a dental bar while physiological saline is injected. Using a dental file, the dental pulp is collected and recovered in a medium. Meanwhile, it is preferable that a person who is a relative within the second degree with respect to a recipient is used as a donor (for example, grandparents are recipients and grandchildren are donors).

### (2) Enzyme Treatment and Inoculation of Cells

The recovered dental pulp is reacted with collagenase and/or dispase. For example, 3 mg/ml of collagenase and 4 mg/ml of dispase are added to a culture medium and left for 1 hour at 37° C. After such enzyme treatment, the culture medium is passed through a cell strainer and contaminating components are removed. The cells are washed and seeded in a culture container. The culture container is transferred into an incubator and cultured (37° C., 5% CO₂). Meanwhile, as the culture liquid, for example, DMEM (Dulbecco's Modified Eagle's Medium) to which blood serum and the like have been added can be used. Specific examples of the culture liquid include DMEM to which fetal bovine serum (20%), penicillin (100 U/ml), streptomycin (100 μg/ml) and amphotericin B (0.25 μg/ml) have been added. As the culture container, a culture dish, a conical flask or the like is used. In this case, serum is not necessarily required.

### (3) Selectively Culturing of Adherent Cells

(12-1) In this step, firstly, adherent cells are selected. The adherent cells can be selected by removing suspended components. Suspended cells can be removed easily by replacing a medium with a new one. Specifically, a part of or substantially all the medium is removed by sucking, and subsequently, a new medium is poured into a culture flask. Thus, a part of or substantially all the medium is replaced. This replacement of media may be repeated a plurality of times. In order to wash and remove the suspended components sufficiently, it is preferable that the replacement of media is carried out three to four times per week.
(12-2) Adherent cells having adherentness (cells attached to a culture flask), which remain after removing suspended components, are further cultured. The culture herein can be carried out under the same conditions as those in step (2). During culture, the culture medium is appropriately replaced with a new one. For example, the culture medium is replaced with a new one every three days.
(12-3) At the stage in which cells are proliferated to some extent, passage culture (expansion culture) may be carried out. For example, when cells are subconfluent (in a state in which about 70% of the surface of the culture vessel is occupied by cells) or confluent by visual observation, cells are peeled off from the culture flask and recovered. Then, they are plated in a culture flask filled with a culture medium. Passage culture may be repeated. For example, passage culture is carried out once to three times so that cells are proliferated to the necessary number of cells (for example, about 1×10⁷ cells/ml). Note here that cells can be peeled off from the flask by a routine method such as treatment with trypsin.

### (3) Differentiation

In this step, proliferated cells are subjected to induction treatment so as to differentiate into a certain cell lineage. If a composition for autotransplantation or allotransplantation for reconstruction of bone tissue is to be prepared, a treatment of differentiation is conducted so as to differentiate into osseous cells. In a typical technique, the differentiation to an osseous cell is promoted by adding three kinds of additive agents, that is, dexamethasone (Dex), β-sodium glycerophosphate (β-GP), and L-ascorbic acid 2-phosphate (AsAP) into a culture medium (or replacing a medium with a medium containing such additive agents (replacing a medium with a bone inducing medium)). As the addition amount of such additive agents, for example, dexamethasone is about 10 mM, β-sodium glycerophosphate is about 10⁻⁸M, and L-ascorbic acid 2-phosphate is about 0.05 mM. During differentiation-inducing time, the medium is appropriately replaced with a new one. For example, the medium is replaced with a new one every three days. Culture for inducing differentiation continues for, for example, one day to 14 days.

### (5) Recovering of Cells

Next, the cells after differentiation induction are recovered. The cells can be recovered by peeling the cells from the culture container by trypsin treatment or the like, and subjecting the cells to centrifugation treatment. Using the cells recovered as above, a transplantation material is prepared. Hereinafter an example of a method for preparing a transplantation material is shown.

### (Method for Preparing Transplantation Material)

In the following preparation method, (i) a step of providing a thrombin solution; (ii) a step of preparing platelet-rich plasma (PRP); and (iii) a step of mixing each component and gelating the mixture. Hereinafter, each step is described, respectively.

### (i) Step of Providing Thrombin Solution

In this step, a solution containing a predetermined amount of thrombin is provided. The concentration of thrombin in the thrombin solution is not particularly limited but made to be a concentration at which an appropriate gelation can be achieved in the below-mentioned step (iii). For example, the concentration of the thrombin solution is determined so that the thrombin solution contains 100 U/ml to 10000 U/ml of thrombin. Preferably, the thrombin concentration is made to be about 1000 U/ml. From the viewpoint of safety and immunological rejection, human thrombin is preferably used. As the human thrombin, for example, Thrombin-YOSHITOMI (registered trademark) can be used. Alternatively, human thrombin prepared from the autologous blood may be used. By acting thrombin in the presence of calcium ions, fibrin is generated from fibrinogen in platelet-rich plasma (PRP) and coagulated (gelated). Therefore, if a thrombin solution containing a calcium ion is provided, when the thrombin solution and PRP are mixed (step (iii)), it is not necessary to add calcium ions. For example, it is preferable that a thrombin solution is prepared as a 5% to 25% calcium chloride solution. It is further preferable to use a thrombin solution prepared as an about 10% calcium chloride solution.

### (ii) Step of Preparing Platelet-Rich Plasma (PRP)

(17-1) In this step, platelet-rich plasma (PRP) is prepared from blood separated from a living body. (17-2) PRP can be prepared in accordance with the Nisseki PC (platelet concentrated) collection method. Specific example of the method for preparing PRP is described hereinafter. Firstly, an anticoagulant agent such as sodium citrate is added to the collected blood and the collected blood is stood still for a predetermined time at room temperature, followed by subjecting it to centrifugation under conditions in which blood cells and buffy coat are separated (for example, at about 1,100 rpm for about 10 minutes). Thus, the blood is divided into two layers. The upper layer is collected and then the remaining blood is further centrifuged at about 2,500 rpm for about 10 minutes. The resultant fragments (Platelet-rich Plasma: PRP) are collected. The method for preparing PRP is not limited to this alone. PRP can be prepared by a method that has been modified if necessary.
(17-3) From the viewpoint of toxicity and immunological rejection, it is preferable that PRP is prepared by using the blood of a recipient him/herself (that is to say, a subject to whom the composition of the present invention is applied). However, PRP may be prepared from allogeneic blood.
(18-1) The number of platelets contained in PRP (concentration rate of platelet) is not generally defined. The plasma containing platelets that are about 150% to about 1500% more than those of the collected blood may be defined as PRP of the present invention.
(18-2) The “platelet concentration rate” of PRP of the present invention is expressed by the following equation.

platelet concentration rate (%)=(average number of platelets in PRP)/(average number of platelets in whole blood as a starting material)×100

(18-3) Therefore, when for example, the average number of platelets in PRP is 1,000,000 and the average number of platelets in whole blood is 300,000, the platelet concentration rate (%) is about 333%. As a result of the previous study, it has been clear that the platelet concentration rate of PRP has a relation with respect to the regeneration effect of tissue. Therefore, in order to obtain the higher regeneration effect, it is preferable to use PRP having a platelet concentration rate in the range from about 150% to about 1500% (generally corresponding to about 240,000 cells/μL to about 6,150,000 cells/μL when converted into the average number of platelets). More preferably, it is preferable to use PRP having a platelet concentration rate in the range from about 300% to about 700% (generally corresponding to about 480,000 cells/μL to about 2,870,000 cells/μL when converted into the average number of platelets).
(18-4) By appropriately adjusting the conditions of the centrifugation when PRP is prepared, it is possible to obtain PRP with a desired platelet concentration rate. For example, when the two-stage centrifugation as mentioned above is carried out and when the first centrifugation is carried out under the conditions of about 500 rpm to about 1500 rpm (for example, 1,100 rpm) for about 5 minutes to about 15 minutes (for example, for about 5 minutes) and the second centrifugation is carried out under the conditions of about 2000 rpm to about 5000 rpm (for example, 2,500 rpm) for about 5 minutes to about 15 minutes (for example, for about 5 minutes), it is possible to obtain PRP having the platelet concentration rate ranging from about 300% to about 700%. It is predicted that the platelet concentration rate of finally obtained PRP varies due to the difference in the blood as the starting material and instruments to be used even if the treatment is carried out under the same condition. The person skilled in the art can find conditions for preparing PRP with a desired platelet concentration rate by modifying the conditions based on the platelet concentration rate of the obtained PRP while considering the above-mentioned conditions.
(18-5) Note here that the measurement of the platelet concentration of PRP can be carried out in accordance with a routine procedure (for example, by using commercially available Sysmex XE-2100 (Sysmex, Tokyo, Japan)).

Platelet concentration rate of PRP used for constructing the composition for autotransplantation or allotransplantation of the present invention is as mentioned above. On the other hand, the platelet concentration of the final composition (the composition for autotransplantation or allotransplantation of the present invention) varies depending upon the platelet concentration rate of PRP and the using ratio of PRP and other components combined with PRP. However, the platelet concentration is for example, about 240,000 cells/μL to about 6,150,000 cells/μL, and preferably about 480,000 cells/μL to about 2,870,000 cells/μL. By containing platelets in such a concentration, an excellent effect of regenerating tissue is obtained. Note here that for example, by using PRP having the platelet concentration rate ranging from about 300% to about 700%, it is possible to adjust the platelet concentration of the final composition to be about 480,000 cells/μL to about 2,870,000 cells/μL.

(iii) Step of Mixing Components and Gelating Mixture
(20-1) In this step, the thrombin solution provided in the step (i), the platelet-rich plasma prepared in the step (ii) and dental pulp stem cells prepared by the method described above are mixed in the presence of calcium ions and the mixture is gelated. In this step, cytokines (BMP, PDGF, bFGF and the like) can also be mixed.

Preferably, when these components are mixed, air is mixed at a predetermined ratio. When air is mixed, the gelation state (fluidity) can be adjusted. Furthermore, when the composition in which air is mixed is transplanted into a living body, with the appropriate amount of air existing in the vicinity of the composition, an environment suitable for cells in the composition to survive and grow can be made. Thus, an excellent tissue regeneration effect can be expected.

(20-2) In the present invention, components are mixed, for example, at the following mixing ratio (based on the volume) and the mixture is gelated.

(a) thrombin solution: total amount of platelet-rich plasma and dental pulp stem cells: air=1:3 to 7:0.1 to 5.0

By mixing the components with the above-mentioned mixing ratio, it is possible to obtain a gel-state composition having an appropriate fluidity from the viewpoint of an operation property and fixity after transplantation as well as exerting an excellent regeneration effect (treatment effect).

(20-3) Herein, the larger the mixing ratio of the total amount of the platelet-rich plasma and dental pulp stem cells becomes and/or the larger the mixing ratio of air becomes, the lower the fluidity of the obtained gel-state composition becomes. That is to say, by manipulating the mixing ratio of (a), the fluidity of the obtained gel-state composition can be adjusted. Specifically, if a composition with relatively low fluidity is needed, among the above-mentioned range of the mixing ratios, the mixing ratio, in which the total amount of platelet-rich plasma and dental pulp stem cells (and/or the mixing ratio of air) is small, may be employed. If a composition with relatively high fluidity is needed, among the above-mentioned range of the mixing ratios, the mixing ratio, in which the total amount of platelet-rich plasma and dental pulp stem cells (and/or the mixing ratio of air) is large, may be employed.
(20-3) Note here that since the thrombin solution, the platelet-rich plasma and the dental pulp stem cells are components originated in a living body, the characteristics may vary to some extent due to the difference in the collection source and the like, and this is thought to affect the gelation state. However, when according to the investigation results to date by the present inventors, the components are mixed in the above-mentioned range of mixing ratio, it is confirmed that a gelation-state composition having an excellent property as mentioned above can be obtained.
(21-1) In one preferable embodiment of the present invention, the mixing ratio of each component is as follows.

(a1) thrombin solution: total amount of platelet-rich plasma and dental pulp stem cells: air=1:4 to 6:0.3 to 3.0

(21-2) By employing this mixing ratio, a gelation-state composition having a desired fluidity can be prepared more reliably.

The concrete mixing ratio of each component is shown as follows.

thrombin solution: total amount of platelet-rich plasma and dental pulp stem cells: air=1:4:1.0

thrombin solution: total amount of platelet-rich plasma and dental pulp stem cells: air=1:5:1.0

thrombin solution: total amount of platelet-rich plasma and dental pulp stem cells: air=1:6:1.0

By mixing each component at the above-mentioned ratio, typically, a composition containing about 1.0×10⁵ to about 1.0×10⁸ cells/ml of dental pulp stem cells can be obtained. According to such a composition, when it is applied to a tissue defect portion, an excellent tissue regeneration effect can be expected.

### EXAMPLES

### A. Study on Properties of Dental Pulp Stem Cells

### 1. Method

### (1) Growth Test

Dental pulp stem cells were prepared from a deciduous tooth and a permanent tooth, and the growth abilities were compared. First, a deciduous tooth which naturally dropped off or was extracted, and a permanent tooth after extraction were soaked in a solution of chlorohexidine or isodine, and recovered in a culture medium. Then, the deciduous tooth and permanent tooth were divided using a dental bar while physiological saline was injected. Then, a dental pulp was collected using a dental file and recovered in a medium. An enzyme treatment was performed using 3 mg/ml of collagenase and 4 mg/ml of dispase at 37° C. for 1 hour, and the cells were passed through a cell strainer to remove contaminating components. The cells were washed and thereafter seeded on a culture dish, and cultured in an incubator at 37° C., 5% CO₂. DMEM to which fetal bovine serum (20%), penicillin (100 U/ml), streptomycin (100 μg/ml) and amphotericin B (0.25 μg/ml) had been added was used as a culture liquid.

Survival of the cells was confirmed, and thereafter the medium was replaced. Subsequently, the medium was replaced on every three days, and passage was carried out at the timepoint when the cells reached confluent.

The growth abilities of the deciduous tooth dental pulp stem cells and permanent tooth dental pulp stem cells obtained as above were compared using a BrdU cell growth assay kit.

### (2) Flow Cytometry

Expression of various surface markers in the dental pulp stem cells derived from deciduous teeth was examined by flow cytometry (FCM).

### 2. Results

### (1) Growth Ability

Deciduous tooth dental pulp stem cells showed fine growth ability, and became confluent state on the 21^{st} day from initiation of culturing (FIG. 1, lower left). Furthermore, fine growth ability was maintained after passage (FIG. 1, lower right).

On the other hand, the growth ability of the deciduous tooth dental pulp stem cells is higher than that of the permanent tooth dental pulp stem cells (FIG. 2). In addition, the growth ability of the dental pulp stem cells exceeds the growth ability of the bone marrow stem cells (FIG. 2, left).

### (2) Surface Marker

It was found that the cells were CD13 positive, CD29 positive, CD44 positive, CD73 positive, CD105 positive, CD146 positive, CD14 negative, CD31 negative, CD34 negative and CD45 negative (FIGS. 3 and 4).

### B. Transplantation Experiment 1

Using canine animal models, the bone forming ability of the dental pulp stem cells was evaluated.

### 1. Method

### (1) Canine Animal Model

After tooth extraction, bone deficits were formed on both sides of the lower jaw so that the deficits could be vertical to the outer side cortex using a trephine bar having a diameter of 10 mm. A transplantation material (test group) which was prepared by using canine permanent tooth dental pulp stem cells and canine deciduous tooth dental pulp stem cells (deciduous tooth dental pulp stem cells were for allotransplantation), and a transplantation material (control group) which was prepared by using PRP and canine bone marrow stem cells (dMSCs) were transplanted on the thus-formed bone deficit sites, and bone formation was observed.

### (2) Preparation of Dental Pulp Stem Cells

Dental pulp stem cells were collected by a method similar to the method described in 1. (1) of the above-mentioned “Study on properties of dental pulp stem cells”, and cultured. Dental pulp stem cells were collected from deciduous teeth of a puppy in a similar method, and cultured (deciduous tooth dental pulp stem cells). Note that, in order to induce differentiation to osteoblasts, the cells were cultured in wet atmosphere of 95% air and 5% CO₂ at 37° C. by using a medium to which three kinds of additives, i.e., dexamethasone (Dex), sodium β-glycerophosphate (β-GP) and L-ascorbic acid diphosphate had been added. After the culture, the cells were treated with tripsin and used for the preparation of transplantation materials.

### C. Transplantation Experiment 2

Using canine animal models, the bone forming abilities of the permanent tooth dental pulp stem cells (autotransplantation) and deciduous tooth dental pulp stem cells (allotransplantation) were compared to that of bone marrow stem cells (MHCs) and evaluated.

### 1. Method

### (1) Canine Animal Model

After tooth extraction, bone deficits were formed on both sides of the lower jaw so that the deficits could became vertical to the outer side cortex using a trephine bar having a diameter of 10 mm. Deficits only (control), a transplantation material which was prepared by using PRP and canine permanent tooth dental pulp stem cells (autotransplantation) and a transplantation material which was prepared by using PRP and canine deciduous tooth dental pulp stem cells (allotransplantation), and a transplantation material which was prepared by using PRP and bore marrow stem cells were transplanted on the thus-formed bone deficit sites, and bone formation was observed.

### (2) Preparation of Dental Pulp Stem Cells

Dental pulp stem cells were collected from permanent teeth of a parent dog in a method similar to the method described in 1. (1) of the above-mentioned “Study on properties of dental pulp stem cells”, and cultured (permanent tooth dental pulp stem cells). Dental pulp stem cells were collected from the deciduous teeth of a puppy in a similar manner, and cultured (deciduous tooth dental pulp stem cells). Note that, in order to induce differentiation to osteoblasts, the cells were cultured in wet atmosphere of 95% air and 5% CO₂ at 37° C. by using a medium to which three kinds of additives, i.e., dexamethasone (Dex), sodium β-glycerophosphate (β-GP) and L-ascorbic acid diphosphate (AsAP) had been added. After the culture, the cells were treated with tripsin and used for the preparation of transplantation materials.

### (3) Preparation of Bone Marrow Stem Cells

Bone marrow stem cells were obtained by needling the Iliac crest bone marrow of a dog, and isolated according to an already-reported method (Kadiyala, S., Young, R. G., Thiede, M. A., and Bruder, S. P. Culture expanded canine mesenchymal stem cells possess osteochondrogenic potential in vivo and in vitro. Cell Transplant 6, 125, 1997.). To explain briefly, the cells were cultured in a medium obtained by adding a growth aid (0.5 mL of a penicillin-streptmycin mixture comprising 50 mL of a mesenchymal cell growth additive, 10 mL of 200 mM L-glutamine, and 2.5 units of penicillin and 25 μg of streptmycin) to a low-glucose DMEM. In order to induce differentiation to osteoblasts, the cells were cultured in wet atmosphere of 95% air and 5% CO₂ at 37° C. by using a medium to which three kinds of additives, i.e., dexamethasone (Dex), sodium β-glycerophosphate (β-GP) and L-ascorbic acid diphosphate (AsAP) had been added. After the culturing, the cells were treated with tripsin and used for the preparation of transplantation materials.

### (4) Preparation and Injection of Transplantation Material (Cell/PRP Mixture)

About 50 mL of whole blood was collected from a dog, and put into a centrifugation tube comprising 10 mL of a culture liquid together with a preserving agent-free heparin (250 U/mL). The blood was first subjected to a centrifugation treatment by using a standard laboratory centrifuge (Himac CT, Hitachi, Ltd., Tokyo, Japan) under a condition of 1100 rpm for 5 minutes. Then, the monolayer intermediate layer of yellow plasma (including leptomeninges comprising blood platelets and leucocytes) was collected using a cannula. In order to pelletize the platelet, second centrifugation treatment was carried out under the condition of 2500 rpm for 5 minutes. Then, the plasma supernatant which was platelet-poor plasma (PPP) and contained a relatively small content of cells was removed. The obtained platelet pellet, i.e., leptomeninges/plasma fraction (PRP) was suspended in 5 mL of the residual plasma, and used for a platelet gel. The numbers of the platelets in PRP and PPP were measured by Sysmex XE-2100 (Sysmex, Tokyo, Japan). As a result, the total number of the platelets was 295,000 (in the range from 224,000 to 333,000) on an average value. On the other hand, the number of the platelets in PRP was 1,293,400 (in the range from 935,000 to 1,840,000) on an average value. By these measurement results, it could be confirmed that the platelet could be separated, and it was found that the degree of concentration in PRP was 438% (relative to the number of the platelets in whole blood (100%)). PRP was stored in a general shaker at room temperature up to the time of use.

Powdery bovine thrombin (5,000 units) was dissolved in 5 mL of a 10% solution of calcium chloride in a separate sterilized container. Then, 1.8 mL of PRP, cells (the dental pulp stem cells prepared in (2) (1.0×10⁷ cells/mL) or the bone marrow stem cells prepared in (3) (1.0×10⁷ cells/mL)), and 0.1 mL of air were aspirated into a 2.5 mL syrinde, and 300 μL of a thrombin-calcium chloride mixture was aspirated into a second syrinde (2.5 mL). The cells were suspended again directly in PRP. The above-mentioned two syrindes were connected with a three-way stopcock and alternately mixed in both syringes so that air bubbles could commute between the two syringes. By the effect of thrombin to act on fibrin to form an insoluble gel, the content showed a gel-like viscosity within 5 to 30 seconds. The obtained gel-like compositions (a PRP/dental pulp stem cell mixture, and a PRP/bone marrow stem cell composition) were injected (transplanted) into the bone deficit sites.

### (5) Histological and Tissue Form Metrological Analyses

At 2 weeks, 4 weeks and 8 weeks after transplantation, each transplanted site was cut out (diameter 2 mm) using a trephine bar, and subjected to a histological analysis. The specimens were fixed with a 8% formalin buffer, decalcified (K-CX; Falma, Tokyo, Japan), and subjected to hematoxylin-eosin staining. These specimens were observed under an optical microscope, and bone formation was evaluated.

### 2. Results

### (1) Differentiation of Dental Pulp Stem Cells to Osteoblasts

Bone nodes were observed on the 15^{th} day from initiation of differentiation induction, and increase of the bone nodes over the culture time was observed (FIG. 5). As a result of analysis of the cells on the 26^{th} day from initiation of differentiation induction, expression of an osteoblast marker was confirmed (FIG. 6).

### (2) Comparison and Evaluation of Bone Formability

The hematoxylin-eosin stained images at 2 weeks, 4 weeks and 8 weeks after transplantation are shown in FIGS. 7 to 9, respectively. Increase in the bone tissue over time was observed in those to which the PRP/deciduous tooth dental pulp stem cell mixture was transplanted (deciduous tooth dental pulp group), and also in those to which the PRP/permanent tooth dental pulp stem cell mixture was transplanted (permanent teeth dental pulp group). It is understood that the bone regeneration effects thereof are similar to the effects of those to which a PRP/bone marrow stem cell mixture was transplanted (bone marrow MSCs group). On the other hand, sufficient bone regeneration was not observed in the control group.

### D. Transplantation Experiment 2 (Study on Treatment Effect of Deciduous Tooth Dental Pulp Stem Cells Using Wound Healing Models)

### 1. Purpose of Experiment

That a wound due to an operation or the like is cured without forming a scar is called as scarless healing, and it is an important goal to be aimed at in operations in the fields of surgery including fields of maxillofacial surgery and plastic surgery. At present, however, it is difficult to control scars in many fields including facial surface in operations of cleft lips, traumas or tumors, and a patient bears a great burden in the case where a scar is unfortunately formed. In many cases where scars and the like are formed, the current mainstream treatment method is a method comprising surgical resection. Since wound healing is a complex procedure in which many growth factors and cytokines are involved, it is considered difficult to control healing of a wound by one of these factors in order to heal the wound so as to minimize the saliency of the wound, and no effective method has been reported so far. In order to study the effectivity of dental pulp stem cells for wound healing, an experiment using wound healing models was carried out.

### 2. Experimental Method

### (1) Culturing of Cells

Using human deciduous tooth dental pulp stem cells (hSHED), human oral mucosa-derived fibroblasts (hFibro) and human bone marrow-derived mesenchymal stem cells (hMHCs), the effects on wound healing and formation of scars were compared and evaluated. Since deciduous teeth physiologically drop off in accordance with growth of humans, they are expected to be a more low-invasive source of stem cells.

### (2) Cell Transplantation Experiment

A silicone plate model, which was already reported, was used. As animals, nude mice (KSN/slc) were used. Wounds were formed on two sites in total, one site at the left and one site at the right, on the dorsal region of a nude mouse using an existing biopsy punch (8 mm). The outer diameter of the silicone plate (0.5 mm) was 16 mm and the inner diameter was 8 mm, the silicon plate was attached by using a quick setting adhesive, and the circumference and skin were sutured together using a 4-0 silk thread. The cultured cells (5×10⁶ cells) were mixed with 100 μl of PBS, and transplanted by injection using 30G. Finally, Tegaderm was attached for the purpose of protecting the wounded site. Meanwhile, PBS was transplanted as a control.

### 3. Experimental Results

### (1) Measurement of Morphology of Wounded Site

Change of the surface area of the wounded site over days was photographed using a digital camera, and the surface area of the wounded site was measured. The difference between the cell-transplanted group and control group was observed in 14 days (FIG. 10). This result shows that the deciduous tooth dental pulp stem cells are effective cells for cell-transplantation therapy similarly to mesenchymal stem cells and fibroblasts.

### (2) Immunofluorescence Staining

According to the report that hyarulonic acid, which is an extracellular matrix, temporarily plays an important role with respect to wound healing and increases its level after wounding, it is considered that hyarulonic acid plays an important role in the process of wound healing. Therefore, change in the level of hyarulonic acid was also studied. The experimental animals were sacrificed on the 7^{th} and 14^{th} days after the cell transplantation experiment. Triple staining was performed with nuclear staining by PKH26, hyarulonic acid-binding protein and DAPI which were marked in advance, and the tissues were evaluated. By immunofluorescence staining after 7 and 14 days, hyarulonic acid which seemed to be released from the cells, was confirmed around the transplanted cells (FIGS. 11 and 12).

### (3) Summary

From the above-mentioned results, wound healing effect similar to that of mesenchymal stem cells (MSCs) which is considered to promote wound healing can be expected for deciduous tooth dental pulp stem cells. It is considered that synthesis of hyarulonic acid is involved in the process of wound healing. The above-mentioned results suggest that deciduous tooth dental pulp stem cells and MSCs synthesize hyarulonic acid and promote wound healing. It can be considered that deciduous tooth dental pulp stem cells can be applied to aesthetic improvements such as wrinkles and regeneration of interdental papilla, as well as promotion of healing of scars such as cleft lips and palate, keloid and surgical wounds.

### INDUSTRIAL APPLICABILITY

The present invention enables regeneration of tissues by autotransplantation or allotransplantation. Therefore, the applicable scope is significantly expanded as compared to the case by autotransplantation. As examples of the indications of the composition for autotransplantation or allotransplantation of the present invention, bone diseases (inclusive of periodontal disease, bone deficits (inclusive of bone increase for implant, fissures on jaw, tumor excision site and the like), osteoporosis, bone fracture, ligament rupture, sport trauma and the like), cartilage diseases (knee joint, jaw joint and the like), nerve diseases (nerve regeneration: Alzheimer's disease, Parkinson's disease and the like), skin diseases (applications to keloids and scars), aging disease (anti-aging diseases such as wrinkles and blotches), blood vessel disorders (regeneration of blood vessels and the like) are envisaged. Furthermore, utilization in regeneration of hair can also be expected.

According to the composition for autotransplantation or allotransplantation of the present invention, an effect that cells which were previously discarded as medical waste can be utilized is also exhibited.

The present invention is not limited to the description of the above exemplary embodiments and Examples. A variety of modifications, which are within the scopes of the following claims and which are easily achieved by a person skilled in the art, are included in the present invention.

Contents of the theses, Publication of Patent Applications, Patent Publications, and other published documents referred to in this specification are herein incorporated by reference in its entity.

1. A composition for autotransplantation or allotransplantation, comprising a dental pulp stem cell from a deciduous tooth or a permanent tooth.

2. The composition for autotransplantation or allotransplantation according to claim 1, wherein the dental pulp stem cell is CD13 positive, CD29 positive, CD44 positive, CD73 positive, CD105 positive, CD146 positive, CD14 negative, CD34 negative and CD45 negative cell.

3. The composition for autotransplantation or allotransplantation according to claim 1, which is characterized by comprising the dental pulp stem cell by about 1.0×10³ to about 1.0×10⁸ cells/ml.

4. The composition for autotransplantation or allotransplantation according to claim 1, comprising a platelet-rich plasma.

5. The composition for autotransplantation or allotransplantation according to claim 1, which is characterized by that the dental pulp stem cell is suspended in a physiological saline or a phosphate-buffered physiological saline.

6. The composition for autotransplantation or allotransplantation according to claim 1, comprising a cytokine.

7. The composition for autotransplantation or allotransplantation according to claim 1, wherein the composition is used for regeneration of a bone tissue, a cartilage tissue, a nerve tissue, a skin tissue, a hair tissue, a periodontal tissue or a blood vessel tissue.

8. A method for regenerating a tissue, wherein the composition for autotransplantation or allotransplantation according to claim 1 is injected in, embedded in, filled in or applied on a tissue-defective site.
